(19) United States
(12) Patent Application Publication
(10) Pub. No.: US 2013/0083311 A1
(43) Pub. Date: Apr. 4, 2013

(54) MICROFLUIDIC SYSTEM FOR OPTICAL MEASUREMENT OF PLATELET AGGREGATION

(71) Applicants: MELISSA LI, Atlanta, GA (US); Craig Richard Forest, Atlanta, GA (US); David Nelson Ku, Atlanta, GA (US)

(72) Inventors: MELISSA LI, Atlanta, GA (US); Craig Richard Forest, Atlanta, GA (US); David Nelson Ku, Atlanta, GA (US)

(21) Appl. No.: 13/632,953

(22) Filed: Oct. 1, 2012

Related U.S. Application Data
(60) Provisional application No. 61/540,767, filed on Sep. 29, 2011.

(51) Int. Cl. G01N 33/49

(52) U.S. Cl. 356/39

(57) ABSTRACT

Described herein in several embodiments are a system, method and apparatus for measuring platelet thrombus volume of a blood sample. In some embodiments, the present invention comprises a microfluidic apparatus having a blood input that receives the blood sample and a plurality of flow channels. In some embodiments of the present invention, each flow channel has a non-stenotic region that receives a portion of the blood sample from the blood input and a stenotic region for creating a discrete initial shear rate in the flow channel. The initial shear rate in the flow channel can be from approximately 500 s⁻¹ to approximately 13000 s⁻¹. The stenotic region, or a narrowing or stricture of the flow channel passageway, is used to simulate a partial blockage of a blood vessel that can result in thrombosis within the blood vessel.

### CROSS REFERENCE TO RELATED APPLICATION AND PRIORITY CLAIM

The present application claims the benefit of U.S. Provisional Patent Application Ser. No. 61/540,767 filed 29 Sep. 2011, and entitled “Microfluidic System for Simultaneous Optical Measurement of Platelet Aggregation at Multiple Shear Rates in Whole Blood,” which is hereby incorporated by reference in its entirety as if fully set forth below.

### TECHNICAL FIELD

The presently disclosed subject matter relates generally to the measurement of platelet aggregation.

### BACKGROUND

Thrombosis is the pathological formation of blood clots due to platelet aggregation that initiates strokes and heart attacks, the leading causes of death in developed nations. Instrumentation capable of characterizing and exploring treatments for platelet aggregation thus has the potential for major impact. Current instrumentation methods focus on single blood samples or flow conditions, but comparative and comprehensive studies involving multiple, pathologically relevant test conditions (e.g., shear rate, drug concentration, hematocrit, channel stenosis) require instrumentation that overcomes limitations on throughput, analysis time, cost and volume while minimizing sample handling.

Both biological and mechanical factors play primary roles in pathological, high shear platelet aggregation known as thrombosis—currently the leading cause of death in developed nations. Biological factors, including soluble agonists, vessel wall composition, and cell surface changes, have been well characterized. In contrast, the effects of mechanical factors including shear stress, hemodynamics, shear rate, and vessel morphology, remain poorly characterized despite ample evidence of their influence. Platelets have long been known to aggregate in response to pathological shear rates exceeding 4000 s⁻¹ in contrast to the mean physiological range estimated at 500-1500 s⁻¹. This behavior is significant because such elevated shear rates are commonly observed in clinical cases of coronary artery disease and atherosclerosis, conditions which affect more than 17 million Americans.

Furthermore, studies have shown the importance of locally constricted “stenotic” blood vessel morphologies in forming thrombosis both in vivo and in vitro models. However, these experiments utilize single blood samples at single flow conditions, thus precluding systematic, comparative and comprehensive analyses. Current methods have been limited by one or more of the following parameters: poor relevance to physiological flow, need for external cell labeling, platelet fractionation and washing for imaging and analysis, non-pathologically relevant shear rates, small measurement area limited by microscopy, and large volumes of blood required. Additionally, many in vitro models have not attempted to address aspects of specific clinical pathologies relevant to thrombosis including high shear rates and stenotic morphology. Conventional methods for platelet function analysis are typically only able to perform testing on single samples at a single shear rate under flow conditions which are not biologically relevant.

Measuring platelet aggregation using in vitro devices has also been challenging. The formation of a thrombus occurs in three phases: (I) initial adhesion of platelets to a substrate, (II) aggregation of platelets to bind to other platelets, and (III) final stabilization and contraction of the platelets into a solid mass that occludes flow. Current studies are often conducted only on the initial adhesion phase using fluorescence microscopy and are not well suited to volumetric measurements in real time. Microscopy methods are limited by small measurement areas, the need for image post-processing, and often require sample pre-treatment. Commercial methods that do measure the time to full flow occlusion have limitations previously described.

### SUMMARY

Described herein in several embodiments are a system, method and apparatus for measuring platelet thrombus volume of a blood sample. In some embodiments, the present invention comprises a microfluidic apparatus having a blood input that receives the blood sample and a plurality of flow channels. In some embodiments of the present invention, each flow channel has a non-stenotic region that receives a portion of the blood sample from the blood input and a stenotic region for creating a discrete initial shear rate in the flow channel. The initial shear rate in the flow channel can be from approximately 500 s⁻¹ to approximately 13000s⁻¹.

The stenotic region, or a narrowing or stricture of the flow channel passageway, is used to simulate a partial blockage of a blood vessel that can result in thrombosis within the blood vessel, such as an atherosclerotic plaque. The stenotic regions of one or more of the flow channels can be the same to provide for redundant testing, different dimensions to test multiple shear rates, or a combination thereof. In some embodiments of the present invention, the diameter of the stenotic region at its most narrow is at least 50% smaller than the average diameter of the non-stenotic region of one or more of the flow channels. In still further embodiments of the present invention, the flow channel non-stenotic region diameter is from approximately 2.54 micrometers to approximately 6 millimeters and the length of the flow channel is from approximately 1.0*10⁻⁵ to 4.0*10⁻² meters in length.

In some embodiments of the present invention, the present invention further comprises an optical system for measuring an increase in radiation transmission as platelets in the blood sample aggregate in the stenotic regions during shear-mediated thrombosis. In some further embodiments of the present invention, the optical system comprises a light source. In some examples of the present invention, the light source is a monochromatic light source. In still further embodiments of the present invention, the light source is a 650 nm diode laser that is mated with a charge coupled device (CCD). In some embodiments of the present invention, the optical system uses a single silicon diode photo-detector. In additional embodiments of the present invention, the optical system can further comprise an aperture plate having a plurality of holes to make simultaneous measurements. In still further embodiments of the present invention, the optical system can further comprise an optical spatial filter for converting the light source into a plane wave.

Another embodiment of the present invention is a method for measuring platelet thrombus volume of a blood sample, the method comprising introducing the blood sample into a microfluidic apparatus, the microfluidic apparatus comprising a blood input that receives the blood sample, a plurality of flow channels, each flow channel comprising a non-stenotic region that receives a portion of the blood sample from the blood input, and a stenotic region for creating a discrete initial shear rate in the flow channel. In some embodiments of the present invention, the method further comprises measuring an increasing in radiation transmission as platelets in the blood sample aggregate in the stenotic regions during shear-mediated thrombosis using an optical system. In further embodiments of the present invention, the method further comprises passively controlling channel flow resistance downstream of the stenotic region using resistive tubing.

A still further embodiment of the present invention is a microfluidic apparatus for measuring platelet thrombus volume of a blood sample, the apparatus comprising a blood input that receives the blood sample, a plurality of flow channels, each flow channel comprising a non-stenotic region that receives a portion of the blood sample from the blood input, and a stenotic region for creating a discrete initial shear rate in the flow channel from approximately 500 s⁻¹ to approximately 13000 s⁻¹. In further embodiments of the present invention, the apparatus further comprises resistive tubing downstream of one or more of the plurality of flow channels to passively control channel flow resistance downstream of the stenotic region in each of the plurality of flow channels.

The foregoing summarizes only a few aspects of the presently disclosed subject matter and is not intended to be reflective of the full scope of the presently disclosed subject matter as claimed. Additional features and advantages of the presently disclosed subject matter are set forth in the following description, may be apparent from the description, or may be learned by practicing the presently disclosed subject matter. Moreover, both the foregoing summary and following detailed description are exemplary and explanatory and are intended to provide further explanation of the presently disclosed subject matter as claimed.

### BRIEF DESCRIPTION OF THE DRAWINGS

The accompanying drawings, which are incorporated in and constitute a part of this specification, illustrate multiple embodiments of the presently disclosed subject matter and, together with the description, serve to explain the principles of the presently disclosed subject matter; and, furthermore, are not intended in any manner to limit the scope of the presently disclosed subject matter.

FIG. 1 is a top-view illustration of an apparatus for forming and measuring platelet aggregates from an unfractionated blood sample, according to an exemplary embodiment of the present invention.

FIG. 2 is a side-view illustration of an apparatus for forming and measuring platelet aggregates from an unfractionated blood sample, according to an exemplary embodiment of the present invention.

FIG. 3 is a end-view illustration of an apparatus for forming and measuring platelet aggregates from an unfractionated blood sample, according to an exemplary embodiment of the present invention.

FIG. 4 is a top-view illustration of a system for forming and measuring platelet aggregates from an unfractionated blood sample, according to an exemplary embodiment of the present invention.

FIGS. 5a and 5b illustrate the calculated shear rates that an unfractionated blood sample may undergo while moving through a stenotic region of an apparatus for forming and measuring platelet aggregates from an unfractionated blood sample, according to an exemplary embodiment of the present invention.

FIG. 6 is a Monte Carlo simulated relationship of the percentage of 633 nm light transmitted through a platelet thrombus of varying thicknesses within a 250 μm stenosis filled with blood.

FIGS. 7a and 7b show the formation and measurement of platelet aggregation to full occlusion of flow in the microfluidic device using simultaneous measurements of microscope intensity, I_{microscope}(t); light transmission, Iₗₐₛₑᵣ(t); and flow rate, Q(t), at 10000 s⁻¹ initial shear rate.

FIGS. 8a and 8b show shear rates vs. occlusion times for six initial shear rates measured using the optical system and flow rate.

In the drawings, the same reference numbers identify identical or substantially similar elements or acts. To easily identify the discussion of any particular element or act, the most significant digit or digits in a reference number refer to the Figure number in which that element is first introduced.

Any headings provided herein are for convenience only and do not necessarily affect the scope or meaning of the claimed presently disclosed subject matter.

### DETAILED DESCRIPTION OF ILLUSTRATIVE EMBODIMENTS

Although preferred embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the preferred embodiments, specific terminology will be resorted to for the sake of clarity.

It must also be noted that, as used in the specification and the appended claims, the singular forms “a,” “an” and “the” include plural referents unless the context clearly dictates otherwise.

Also, in describing the preferred embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

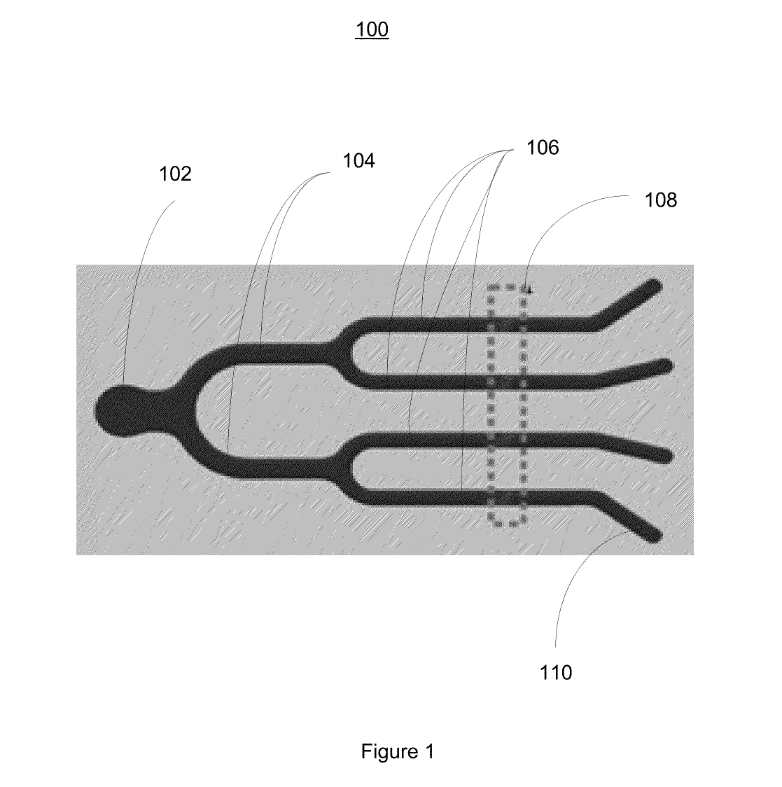

Various embodiments of the present invention are directed to systems, methods and apparati for simulating a partially occluded (or blocked) artery for measuring thrombosis. An exemplary microfluidic apparatus 100 is illustrated in FIG. 1. To use apparatus 100 for measuring thrombosis, apparatus 100 has blood sample input 102 for receiving a blood sample to be tested. The blood can be introduced to apparatus 100 in various ways. For example, a blood sample can be introduced via means of a needle from a syringe (not shown) used to extract the blood sample. In some embodiments, apparatus 100 can be configured to receive the syringe in such a way that the fluid flow from the syringe provides a nominal positive pressure on the blood sample to cause the blood sample to flow from the input 102 to flow channel 104.

Once the blood sample moves from input 102 through flow channel 104, the blood sample is further split into additional flow channels 106. Flow channels 106 comprise stenotic region 108 that has dimensions that simulate a partially occluded artery. In some embodiments, the dimension can be characterized by a narrowing of the diameter of flow channels 106, but other dimensions and flow impediments can be used. As the blood sample flows past stenotic region 108, the blood can impinge on the sidewall of flow channels 106, sometimes causing the aggregation of the platelets onto the sidewall of flow channels 106. This is caused by the shear forces imparted on the blood sample created by the changing internal dimensions of flow channels 106 in stenotic region 108. Apparatus 100 can enable the formation of thrombi at four distinct initial shear rates between 500 and 13000 s⁻¹ from a constant pressure source within corresponding stenotic regions of the branching channels, which is intended to mimic coronary arteries.

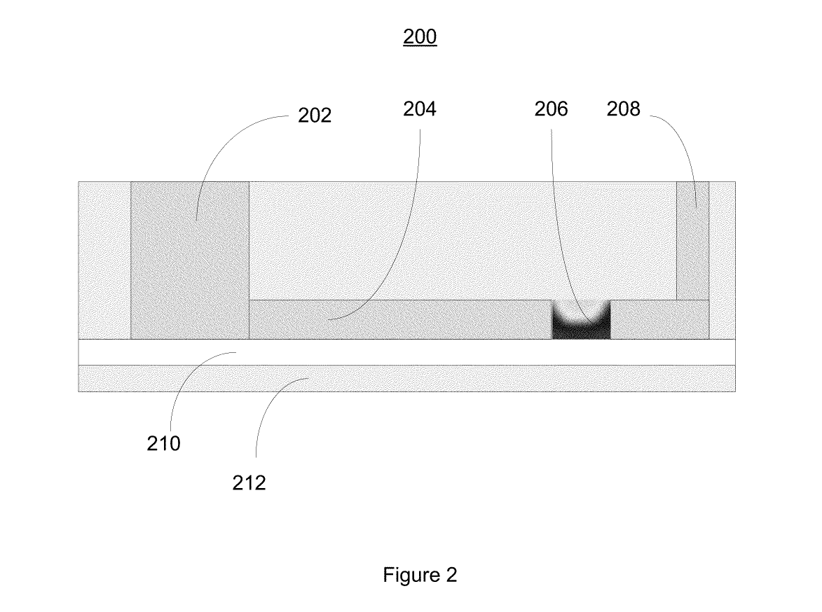

FIG. 2 is a side view of an apparatus for simulating a partially occluded (or blocked) artery for measuring thrombosis. Apparatus 200 has blood input 202 for receiving a sample of blood. A positive pressure is applied to the blood sample (not shown) that causes the blood sample to move from input 202 through flow channel 204. To simulate a partially occluded artery, flow channel 204 has stenotic region 206. In FIG. 2, stenotic region 206 is illustrated as a reduced diameter flow path in relation to the average internal diameter of flow channel 204. Output region 208 is provided to collect the portion of the blood sample flowing through apparatus 200.

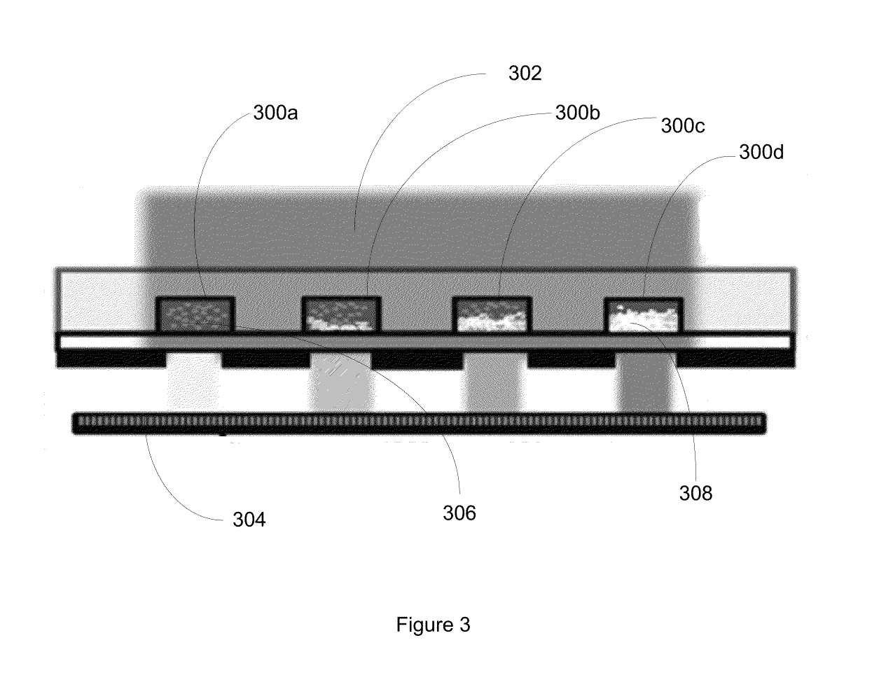

To measure thrombosis, the amount of platelet formation is measured. FIG. 3 is an illustration showing a side view of a microfluidic flow apparatus using an exemplary optical system for measuring platelet aggregation caused by a stenotic region of a flow channel. Illustrated are side views of the stenotic regions of flow channels 300a-d. In this embodiment, the dimensions of stenotic regions vary in size, causing different platelet aggregation characteristics for the same blood sample input.

To measure platelet aggregation, in the embodiment illustrated in FIG. 3, an optical measurement system is used. Light source 302 is directed through the stenotic regions of flow channels 300a-d, which are preferably transparent to various light wavelengths. In some embodiments, the light source may be a laser whose wavelength is 600 nm, an optimal wavelength for measuring platelet concentration, though other wavelengths and light sources can be used. To measure the concentration of platelets, optical sensor 304 is used. Various types of optical sensors can be used. In some embodiments, a CCD is used to measure light intensity transmitted through the stenotic regions of flow channels 300a-d. The transmission of light through blood's components varies. Platelets are less scattering and less absorbing to visible light (250-850 nm) than red blood cells due to differences in their index of refraction and lack of hemoglobin. Therefore, as platelet-rich thrombus accumulates in the stenosis, otherwise filled with blood, light transmitted through it increases. For example, stenotic region of flow channel 300a shows relatively minor amounts of platelet formation, which would allow a weaker input light energy to optical sensor 304, whereas stenotic region of flow channel 300d is shown to have a larger amount of platelet formation, which has a relatively higher input light energy to optical sensor 304.

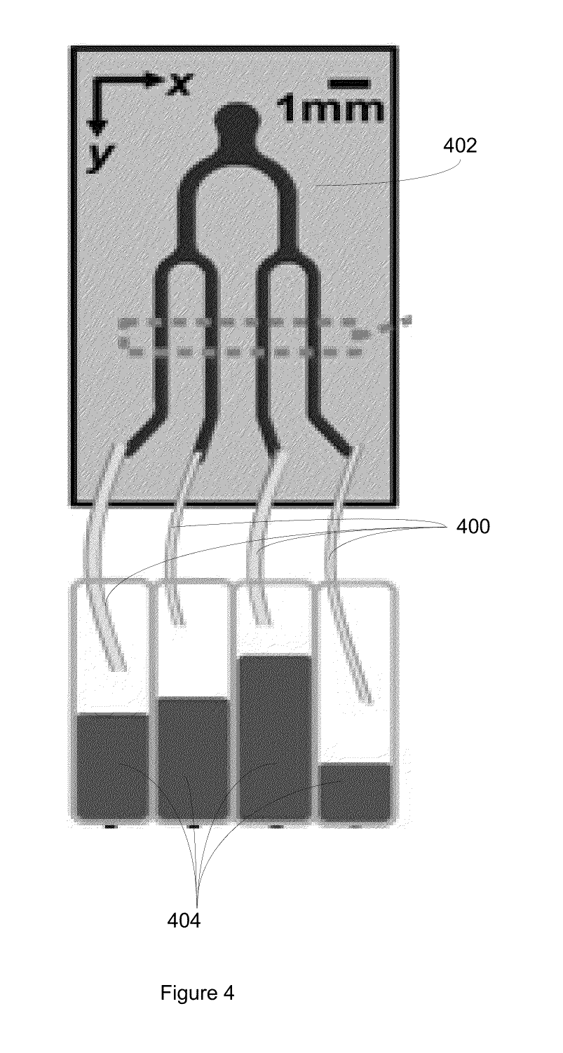

Outlet tubing of various lengths and diameters can be used to induce initial shear rates, as illustrated in FIG. 4. Resistance tubes 400 are connected to their respective flow channels in microfluidic apparatus 402. Tubes 400 can terminate in separate, independent weighing scales 404 to measure volumetric flow rate.

Experimental Results

A system for simulating a partially occluded (or blocked) artery for measuring thrombosis was designed to address shear rates of 500, 1500, 4000, 7000, 10000, and 13000 s⁻¹ within the channels' high-shear stenotic regions. These shear rates were chosen to represent the physiological shear rate range (500-1500 s⁻¹), pathological shear rates (4000 s⁻¹), and high pathological shear rates (10000 s⁻¹ and above). A set of four of these six shear rates were obtained during a single experiment within the branching channels from a common pressure difference of 1400 Pa.

In contrast to constant volumetric flow rate, constant pressure-driven flow seems preferable for the measurement of thrombus growth to full occlusion; as the thrombus reaches full occlusion, constant volumetric flow rate requires large velocities that can cause non-physiologically relevant high forces on the growing platelet thrombus. Blood viscosity, μ, and density, ρ, were assumed constant (valid for shear rate \dot{γ}>6 s⁻¹) at 0.00385 Pa·s and 1.080 g/mL, respectively. As a design consideration, a minimum dimension of 250 μm was chosen to enable observation of rapid platelet accumulation to compare to conventional measurement systems.

Another consideration was the design of the stenosis to reflect pathologically relevant geometry. Thus, the diameter reduction was designed from the channel region to the stenosis region to be 53%, near to the 50% reduction often used as an indicator for surgical intervention in the left main coronary artery. In the channels upstream and downstream of the high-shear stenotic regions, the maximum shear rates did not exceed 1500 s⁻¹, the upper bound of physiological shear rates.

In order to obtain a wide range of shear rates in multiple identical microchannels sharing a single flow input, channel flow resistance was passively controlled downstream of the stenotic region through the use of Tygon tubing (Saint Gobain, 50SHL). Thus, finite volume fluid modeling efforts combined the effects of the channel and tubing resistances. While conventional systems have used on-chip resistive channels for controlling flow rates, the off-chip resistive tubing design of the present invention enables simple shear rate control over two orders of magnitude from an array of identical microchannels, simplifies fabrication, and interfaces easily with weighing scales for flow rate measurement.

To determine shear rate distribution, design the channel geometry, and select resistance tubing, finite volume fluid modeling was performed using ANSYS (Ansys Inc., Canonsburg, Pa.). Modeling was performed using a mesh with volume size of 2 μm minimum side length within the stenosis to approximate the boundary layer region in which a platelet (2-20 μm diameter) binds. Previously mentioned input pressure, viscosity, and density were used to determine shear rate distribution as a function of channel and tubing dimensions. This model was applied to select resistive tubing ranging from L=0.6 m, d=0.8 mm to L=0.3 m, d=2.4 mm.

Light Transmission Through Platelet Thrombus

To design a laser optical system to measure platelet aggregation, a Monte Carlo radiative transport optical modeling tool capable of accounting for the inherent stochastic scattering and absorption of light in blood was applied. The transmission of light through blood's components varies. Platelets are less scattering and less absorbing to visible light (250-850 nm) than red blood cells due to differences in their index of refraction and lack of hemoglobin. Therefore, as platelet-rich thrombus accumulates in the stenosis, otherwise filled with blood, light transmitted through it increases.

The Monte Carlo optical model accounts for the wavelength dependent scattering coefficient, αₛ, absorption coefficient, αₐ, anisotropy of scattering, g, and refractive index, n of both the platelet thrombus and whole blood (hematocrit, Hct=0.41). A published confocal reflectance technique was utilized after confirming its accuracy by measuring known αₛ for Intralipid-20 1% v/v (Sigma-Aldrich, St. Louis, Mo.). Measurements were performed on occlusive platelet thrombi, within two hours of formation. Platelets were labeled with the fluorophore mepacrine to ensure scattering measurements were acquired in the correct location. Mepacrine's 525 nm emission was spectrally excluded from the platelet aggregation measurement wavelength bandpass and its low concentration (<1%) minimized scatting effects.

The laser optical system used for measuring platelet aggregation comprises a 650 nm, 0.9 mW (CVI MellesGriot, Albuquerque, N.M.) laser diode, spatially filtered, along with a 3000 pixel 12-bit linear CCD with pixel dimensions 7 μm×200 μm (Thorlabs, LC1 USB, Newton N.J.). Optical cross-talk between channels was mitigated with sufficient channel spacing and the use of an aperture plate.

Experimental Protocol

Porcine blood samples obtained fresh from a local abattoir were treated immediately with 3.5 Units/mL of unfractionated porcine heparin (Elkins-Sinn Inc., Cherry Hill, N.J.) and were used within five hours of collection. This concentration of heparin has been used by others to prevent clotting in storage reservoirs while allowing thrombus formation under flowing conditions. In contrast, the more widely used citrate requires the addition of ADP to re-activate platelets after its addition. Blood samples were prevented from sedimenting by rotation on a laboratory shaker at about 60 rpm. Prior to experiments, the blood was filtered by flowing it slowly through a 200 μm pore polypropylene mesh (Smallparts, Seattle Wash.) to remove platelet or lipid aggregates that could cause embolic occlusion. To verify the platelet composition of the occlusive thrombus, Carstairs histological staining method was used. To perform Carstairs staining, the PDMS layer containing attached thrombi was excised and preserved with formalin for 48 hours, dehydrated in a series of xylene washes, fixed in paraffin, microtome sectioned into 5 μm slices, and stained. The stain colors platelets blue and red blood cells orange/red. Thrombus volume within the stenosis was estimated by multiplying its cross-sectional area by the height of the stenosis.

Devices were primed with a mixture of 43% glycerol/phospate buffered saline (Sigma Chemical, St. Louis, Mo.), viscosity matched at μ=0.00385 Pa·s prior to flowing blood. Glycerol and blood were pressurized to 1400 Pa for flow into the device with an open, suspended syringe with 132 mm of gravity pressure head. This pressure head was maintained during the course of an experiment with by visually observing changing fluid reservoir height and adding additional fluid with a syringe as necessary, resulting in a pressure uncertainty of ˜30 Pa. The volumetric flow rates were compared between modeled flows, a glycerol solution, and highly heparinized blood (35 Units/mL) to evaluate modeling accuracy.

The microfluidic device was aligned to the laser optical system and microscope using a 3-axis translation stage. Laser intensity transmitted through each of the four stenosis regions, Iₗₐₛₑᵣ(t), was recorded and flow rate, Qₙ(t), was measured by weighing scales with 0.01 g resolution placed at the outlet of each channel n (Adam Equipment, Danbury, Conn.), according to Q=Δm/ρΔt, where m is mass and p is density. White-light microscopy images were acquired with a Zeiss Stemi 2000c microscope under 5.0× magnification and a Motic 2000 CCD camera in a detection method used by others. Hardware control and acquisition were sampled at 1 Hz with 20 ms integration time using Labview (National Instruments, Austin Tex.) and data was analyzed using Matlab (Mathworks, Natick, Mass.). Optical measurements were normalized as [I(t)−Iₘᵢₙ]/Iₘₐₓ and low-pass filtered to produce relative intensity measurements I_{microscope}(t) and Iₗₐₛₑᵣ(t).

Experimental Results

Modeled and experimental flow rates within the microfluidic device were first compared. Next, the system was verified that it able to form occlusive platelet aggregates. An optical model was then used to design and validate the optical system for measuring platelet aggregation and sensing occlusion. Finally, the ability of the integrated microfluidic system to form and measure platelet aggregation to occlusion in parallel with microscopy and flow-based measurements was tested. The system was then applied to measure time and volume to occlusion 108 channel runs amongst 14 porcine samples at varying shear rates and confirmed the formation of platelet-rich thrombi in the channels by histology.

Fluid Modeling

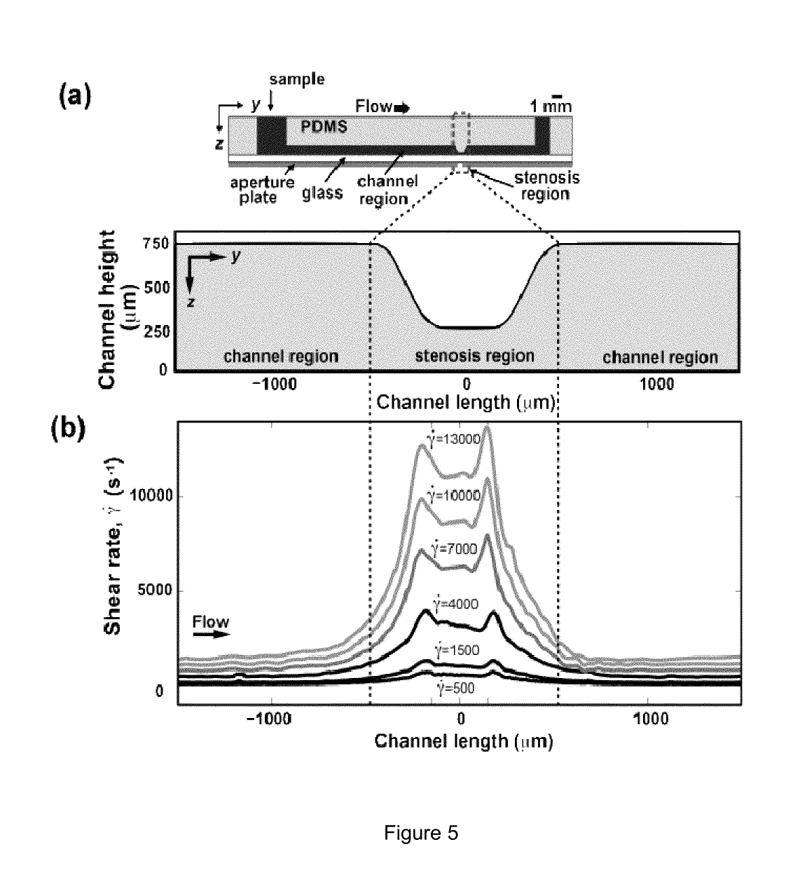

Computational fluid dynamics (ANSYS) was used to quantify shear rate in the test section. The maximum shear rate upstream and downstream of the stenosis region was<1500 s⁻¹, within the physiological range of normal arterial flow. Shear rates were calculated as the average wall shear rate along x-z cross sections. Observing the distribution of shear rates along the channel length revealed the highest shear rate at the entrance of the stenosis, where initiation of platelet aggregation was expected. Flow was laminar with Re=3-27. Additionally, this modeling revealed that a gradual cross-sectional area transition to the stenosis from the non-stenosis channel region reduces flow velocity variation as compared with an abrupt transition, more accurately mimicking physiological conditions and prior in vitro work. FIG. 5 illustrates six shear rate profiles that were computed and obtained experimentally by varying outlet tubing geometry connected to uniform channel geometry (a). The maximum shear rate outside of the stenoses regions is within physiological range <1500 s⁻¹, while maximum shear rate within the stenoses were designed for a variety of physiological and pathological shear rates (500-13000 s⁻1) (b).

Adhered and aggregated platelets reduce the stenosis diameter and correspondingly change the wall shear rate. Applying our fluid modeling, we estimate an exponential increase in shear rate as a function of decreasing cross-sectional area, up to a maximum (2-4 times initial wall shear rate for all shear rates and pressure in this study), after which it rapidly falls to zero. Measured flow rates are compared with modeled flow rates in Table 1. Post-testing microscopy did not show any accumulated platelet mass from the blood flow, as expected.

TABLE 1
Modeled and experimentally verified steady
flow rates in the microfluidic device.

| Shear rate, \dot{γ} | Q_{model} | Q_{glycerol} | Q_{blood} |
|---|---|---|---|
| (s⁻¹) | (μL/s) | (μL/s) | (μL/s) |
| 500 | 1 | 0.5 ± 0.4 | 0.6 ± 0.3 |
| 1500 | 5 | 3 ± 1 | 3 ± 1 |
| 4000 | 15 | 13 ± 1 | 13 ± 1 |
| 7000 | 19 | 17 ± 1 | 17 ± 1 |
| 10000 | 24 | 27 ± 2 | 27 ± 2 |
| 13000 | 40 | 39 ± 7 | 39 ± 3 |

For both glycerol and heparinized blood, flow rates match the model well at all shear rates well. Measured flow rates are shown as averages ±standard deviation. Instrument (weighing scale) measurement uncertainty is 0.3 μL/s.

Thrombus Histology

Thrombus histology results using Carstairs staining method indicate that thrombi formed within the stenosis region of our devices showed high concentrations of platelets with low concentrations of fibrin at high shear rates and red blood cell-rich thrombi at low shear rates, as expected and consistent with previous reports. Volumes of thrombi were estimated at 0.0256 μL (N=7), approximately 55% of the stenosis volume.

Optical Modeling

The experimentally determined scattering coefficient for platelet thrombus is shown along with published optical properties of whole blood and platelets in Table 2 (αₛ=184-203 for N=3).

TABLE 2
Cited and measured optical properties (scattering coefficient, αₛ;
refractive index, n; absorption coefficient, αₐ; anisotropy of
scattering, g)for whole blood and platelet thrombus at 633 nm.

| | αₛ | | αₐ | |
|---|---|---|---|---|
| Sample | (cm⁻¹) | n | (cm⁻¹) | g |
| Blood, Hct 0.41 | 794⁺ | 1.42⁺ | 7.92⁺ | 0.99⁺ |
| Platelet | 193* | 1.35⁺ | 1.21⁺ | 0.96⁺ |
| thrombus | | | | |

*Measured in this work,
⁺Cited from published data

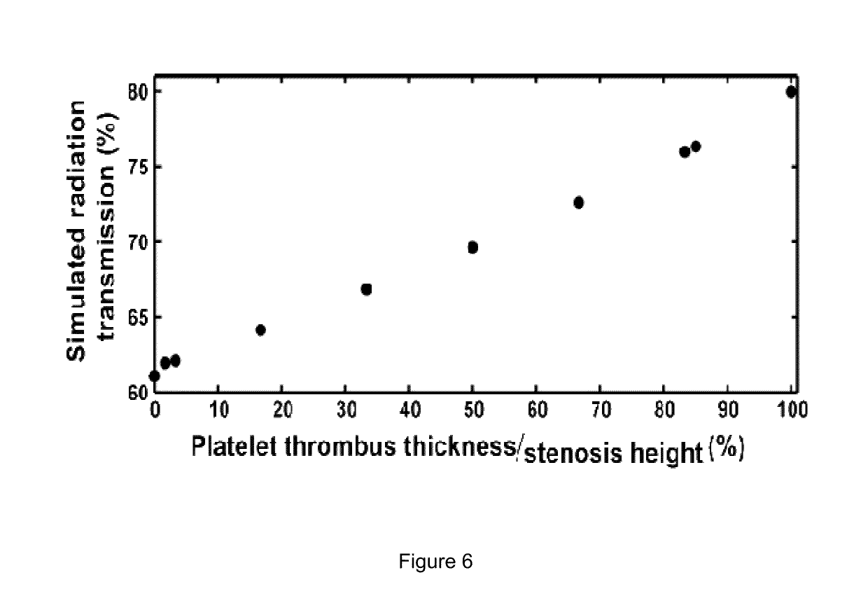

Using the Monte Carlo optical model with the properties of Table 2, the expected increase in transmitted light intensity was calculated as a function of platelet thrombus thickness within the channel. The results, shown in FIG. 6, show a linear increase, up to a 19% change in light transmitted at full occlusion in which the thrombus thickness equals the full channel stenosis height. These results are supported by experimental results, which show increases in absolute light intensity of 24+/−10.2% (N=68) for a thrombus whose thickness spans the entire stenosis height.

Platelet Aggregation Measurements

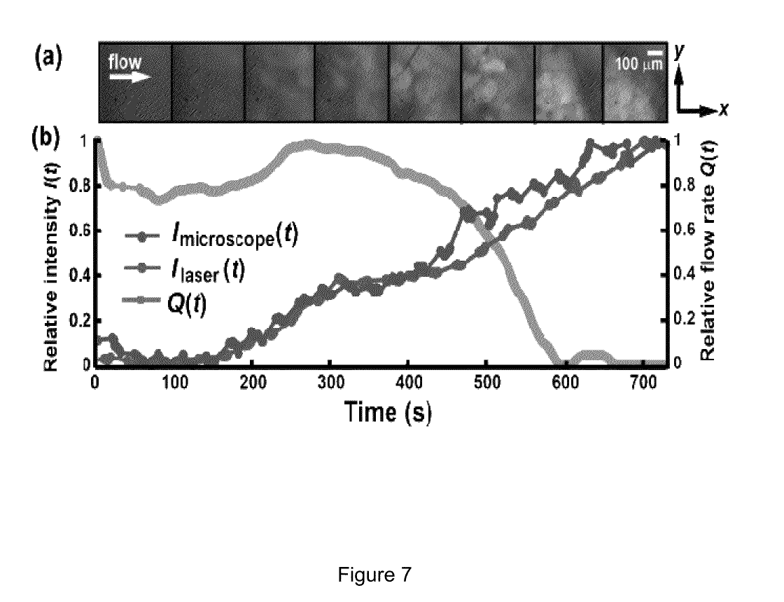

The microfludic system was first used to form and detect platelet aggregation to full occlusion of flow at 10000 s⁻¹ initial shear rate with simultaneous optical (microscope and laser optical system) and flow rate measurements. Results of a typical experiment are shown in FIG. 7. From the time series of raw microscope images of platelets aggregating in the stenosis (FIG. 7a), normalized intensity, I_{microscope}(t), was computed for comparison with the laser optical system intensity transmitted, Iₗₐₛₑᵣ(t) (FIG. 4b). Occlusion times between the methods differ by 3.97%+/−5.1% (N=6) with Pearson product-moment correlation coefficient, or Pearson's r, of 0.94, p<0.01. Both optical methods show rapid platelet accumulation (FIG. 4).

The flow rate measurements, Q(t), shown in FIG. 7b, also show the occlusion time. From this flow rate measurement, the intensity at which occlusion occurs in the laser optical system was calibrated by setting a threshold as a percentage of maximum intensity. Flow rate is nearly constant while platelets accumulate in the stenosis because the stenosis is a small fraction of the overall resistance of the microfluidic channel and associated resistance tubing. From fluid modeling, the resistance of the stenosis was estimated to reach the same order of magnitude as the rest of the system at 28% occlusion, at which point (˜400 s), the flow rate begins to decay logarithmically.

Next, the microfluidic system was applied to whole porcine blood for shear rates spanning physiological to pathological flow conditions. For each porcine blood sample, four shear rates were run simultaneously in a single trial while platelet aggregation to occlusion was measured by the laser optical system only and flow rates were measured by the weighing scales. A trial is defined as a single experiment with one new microfluidic device comprising four channel runs. Up to three trials were run with each sample.

In total, 14 porcine blood samples were run on a total of 27 trials resulting in N=4 channels/device×27 trials=108 channel runs. Of these samples, 5/14 (36%) showed no occlusion at any shear rate or showed emboli at all shear rates, so these were excluded after a single trial. Embolus was defined as decrease in flow rate to less than 50% of initial within 30 s. Of the remaining 9 samples run in 22 trials (N=88 channel runs), none of the channels with initial shear rate of 500 s⁻¹ and 1500 s₋₁ formed occlusive thrombi over 2000 s (N=18). This finding is consistent with previous work which has shown that rapid platelet accumulation is absent with low shear rates (<2000 s ⁻¹). The other N=70 channels were run at a variety of higher shear rates.

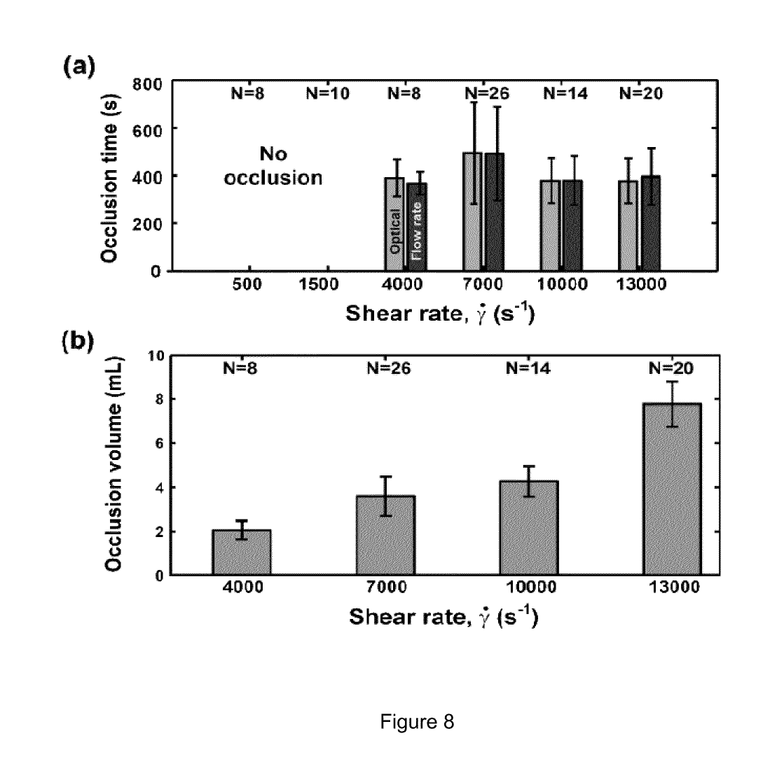

Only two high-shear experiments clotted (one at 4000 s⁻¹, one at 7000 s⁻¹), while in the rest, occlusive thrombi formed at times less than 1200 s (N=68). The times to occlusion for the N=68 channel runs described are shown in FIG. 8a, along with the corresponding standard deviations. The average difference between the optical and flow rate measurement of time to occlusion was 8.4%; the standard deviations of the time to occlusion measurements ranged from 50-212 s. For this range of pathological shear rates (4000-13000 s⁻¹), a statistically significant (p<0.05) difference in the time to occlusion versus shear rate was not observed. This suggests that thrombosis is binary, present above 4000 s⁻¹ and not present below 1500 s⁻¹.

To examine the intra-trial variability of blood clotting behavior, the blood clotting behavior variance between adjacent channels was measured in the same trial with the same blood on the same device at the same shear rate. In 14 trials with duplicate shear rates channel runs (D=14 sets), the average standard deviation of the clotting time, σ, for the sets. The average across all shear rates (D=14) was σ=75 s. The average at specific shear rates was, for \dot{γ}=4000 s⁻¹, σ=30 s (D=2); for \dot{γ}=7000 s⁻¹, σ=99 s (D=9); and for \dot{γ}=10000 s⁻¹, σ=34 s (D=3).

To study the inter-trial variability, the blood clotting behavior variance between successive trials was measured with the same blood at the same shear rate. 9 triplicate channel runs were ran (T=9 sets) and computed the average standard deviation of the clotting times for the sets. The average across all shear rates (T=9) was σ=106 s. The average at specific shear rates was, for \dot{γ}=7000 s⁻¹, σ=155 s (T=3); for \dot{γ}=10000 s⁻¹, σ=61 s (T=2); and for \dot{γ}=13000 s⁻¹, σ=91 s (T=4)). Since the intra- and inter-trial variability measured were similar, occlusion time measurements across different trials and channels at common shear rates were comparable. Similarly, the volume, V, of blood required to form occlusive thrombus at each shear rate are determined (V=m/ρ), shown in FIG. 8b. The volume required are significantly different (p<0.05), save the 7000 s⁻¹ to 10000 s⁻¹ comparison.

Numerous characteristics and advantages have been set forth in the foregoing description, together with details of structure and function. While the invention has been disclosed in several forms, it will be apparent to those skilled in the art that many modifications, additions, and deletions, especially in matters of shape, size, and arrangement of parts, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

What is claimed:

1. A system for forming and measuring platelet aggregates from an unfractionated blood sample, the system comprising:
a microfluidic apparatus comprising:
a blood input that receives the blood sample;
a plurality of flow channels, each flow channel comprising:
a non-stenotic region that receives a portion of the blood sample from the blood input; and
a stenotic region for creating a discrete initial shear rate in the flow channel; and
an optical system for measuring an increase in radiation transmission as platelets in the blood sample aggregate in the stenotic regions during shear-mediated thrombosis.

2. The microfluidic apparatus of claim 1, further comprising resistive tubing downstream of one or more of the plurality of flow channels to passively control channel flow resistance, and thus shear flow within the stenosis, located downstream of the stenotic region in the one or more of the plurality of flow channels.

3. The microfluidic apparatus of claim 1, wherein the initial shear rate in the flow channel is from approximately 500 s⁻¹ to approximately 13000 s⁻¹.

4. The system of claim 1, wherein the optical system comprises a light source.

5. The system of claim 4, wherein the light source is a monochromatic light source.

6. The system of claim 4, wherein the optical system further comprises an aperture plate having a plurality of holes to make simultaneous measurements.

7. The system of claim 4, further comprising a light sensor.

8. The system of claim 7, wherein the light sensor is a single silicon diode photo-detector.

9. The system of claim 4, further comprising an optical spatial filter for converting the light source into a plane wave.

10. A method for forming and measuring platelet aggregates from an unfractionated blood sample, the method comprising:
introducing the blood sample into a microfluidic apparatus, the microfluidic apparatus comprising:
a blood input that receives the blood sample;
a plurality of flow channels, each flow channel comprising:
a non-stenotic region that receives a portion of the blood sample from the blood input; and
a stenotic region for creating a discrete initial shear rate in the flow channel; and
measuring an increasing in radiation transmission as platelets in the blood sample aggregate in the stenotic regions during shear-mediated thrombosis using an optical system.

11. The method of claim 10, further comprising passively controlling channel flow resistance downstream of the stenotic region using resistive tubing.

12. The method of claim 10, wherein the initial shear rate in the flow channel is from approximately 500 s⁻¹ to approximately 13000 s⁻¹.

13. The method of claim 10, wherein the optical system comprises a light source.

14. The method of claim 13, wherein the light source is a monochromatic light source.

15. The method of claim 10, further comprising making simultaneous measurements, wherein the optical system further comprises an aperture plate having a plurality of holes.

16. The method of claim 10, wherein the optical system further comprises a light sensor.

17. The method of claim 16, wherein the light sensor is a single silicon diode photo-detector.

18. The method of claim 10, wherein the optical system further comprises an optical spatial filter for converting the light source into a plane wave.

19. A microfluidic apparatus for use in forming and measuring platelet aggregates from an unfractionated blood sample, the apparatus comprising:
a blood input that receives the blood sample;
a plurality of flow channels, each flow channel comprising:
a non-stenotic region that receives a portion of the blood sample from the blood input; and
a stenotic region for creating a discrete initial shear rate in the flow channel from approximately 500 s⁻¹ to approximately 13000 s⁻¹; and
resistive tubing downstream of one or more of the plurality of flow channels to passively control channel flow resistance downstream of the stenotic region in each of the plurality of flow channels.

20. The apparatus of claim 19, wherein the diameter of the stenotic region at its most narrow is at least 50% smaller than the average diameter of the non-stenotic region of one or more of the plurality of flow channels.

21. The apparatus of claim 19, wherein the non-stenotic region of at least one of the plurality of flow channels has an internal diameter from approximately 2.54 micrometers to approximately 6 millimeters.

22. The apparatus of claim 19, wherein at least one of the plurality of flow channels has a length of from approximately 1.0*10⁻⁵ to 4.0*10⁻² meters.
